(19) United States
(12) Patent Application Publication
(10) Pub. No.: US 2007/0276365 A1
(43) Pub. Date: Nov. 29, 2007

(54) Spinal implants and devices and methods for their controlled insertion

(76) Inventors: Yong Song, Fremont, CA (US); Richard Helkowski, Redwood City, CA (US); Raj Ganpath, Mountain View, CA (US)

Correspondence Address: WHITE & CASE LLP;PATENT DEPARTMENT, 1155 AVENUE OF THE AMERICAS, NEW YORK, NY, 10036, US

(21) Appl. No.: 11/747,206

(22) Filed: May 10, 2007

Related U.S. Application Data
(60) Provisional application No. 60/799,484, filed on May 10, 2006.

(51) Int. Cl. A61B 17/56

(52) U.S. Cl. 606/60

(57) ABSTRACT

Devices and methods for inserting implants for treatment of spinal tissue reduce the profile of the implant for purposes of delivery, permitting delivery of larger implant retention structures, while tending to reduce trauma to tissue and to permit precise axial and lateral positioning of the implant and/or an associated retention structure. Other advantageous features according to the present invention may include delivery of an implant while maintaining at least a substantial portion of low-column-strength portions of the implant in tension, reconfiguring the implant to permit insertion through a diameter significantly smaller than an operative dimension of the implant when in a retention configuration following insertion, and an ability to maneuver, i.e., advance, rotate, position and reposition, the implant, by the practitioner which, among other benefits, can reduce the chances for expulsion of the implant

### CROSS-REFERENCE TO RELATED APPLICATIONS

This application claims priority benefit, under 35 U.S.C. § 119(e), of U.S. Provisional Patent Application 60/799,484, filed May 10, 2006, the contents of which application are hereby incorporated by reference in their entirety.

### FIELD OF THE INVENTION

The present invention relates generally to the field of medical delivery devices and in particular to devices and methods useful in the treatment of spinal disc conditions.

### BACKGROUND OF THE INVENTION

Lumbar discectomy is the most frequently performed operative spine procedure. MRI studies have shown that 20% of patients experience recurrent disc herniation 6 months following lumbar discectomy. Reported clinical outcomes of surgical discectomy have also shown comparable success rates ranging from 40%-90%. The unhealed annulus defect created via an annulotomy during discectomy procedure is considered to be a factor in the lower success rates. Although implants have been considered for treatment of annulus or other spinal defects, there remains a need for controlled insertion and placement of such implants.

### SUMMARY OF THE INVENTION

Embodiments of the disclosed invention provide devices and methods for manual, controlled insertion and retention of an implant or other support body into a desired location, such as in an intervertebral disc. In embodiments of one of its aspects, an implant having dimensions significantly larger than a defect or other point of entry in such a disc may be configured for delivery to occupy a diameter less than or equal to that of the point of entry, facilitating its insertion. The implant may, for example, have a dimension (e.g., of a retention portion of the implant) that is three times, or other multiple, wider than the point of entry, since the invention provides devices and methods permitting the implant to be reconfigured to minimize its profile during insertion. In embodiments of another of its aspects, the configuration or orientation of an implant may be controlled by application of forces at different points of the implant in order to permit adjustment of the implant after insertion to increase its ability to be retained, such as in the nuclear space of an intervertebral disc. In particular embodiments, the selection of points of application of the maneuvering forces, which may comprise features such as through holes, recesses and the like, are selected to facilitate such maneuvering, re-orientation and/or reconfiguration. Application of relative motion for manually adjusting the device, i.e., through the motion of fingers on a trigger relative to the palm of a hand on the handpiece of the device, enables the user to achieve more precise movement near a more distant, foreign body, such as the disc, than would be possible in reliance on absolute movement alone and absent such relative manual movement.

One aspect of the present invention concerns the management of an implant's configuration to reduce not only its profile in relevant ways to facilitate delivery, but to reduce the amount of space needed to accommodate it. The amount of volume required for rotating an object, which is considerably larger than the aperture through which it is inserted, will be the sum of the volume of the object and the volume required to sweep the angle through which rotation will occur. By constraining rotation of the implant, according to the present invention, such as through constraining a rotation suture as described below, rotation is induced along with advancement of the device, thereby advancing and rotating the device simultaneously. The point in time during operation at which the rotation will start can be controlled by varying the slack, e.g., in the rotation suture. This, in turn, results in reducing the volume requirements for rotation, which translates to removing comparatively less volume of nucleus material from the disc space.

Other embodiments of the invention provide a means for preparing the insertion component to be utilized in the body at the point of use. According to aspects of the invention, an insertion device may at least partially compress material to be used as, or as part of, a spinal implant just prior to delivery, giving the implant its functional shape at this later point in time.

In an embodiment of another aspect of the present invention, an insertion device consists of a displacement component progressing implantable material through the spinal disc region and into its ultimate location in a minimally disruptive manner, by distributing the contact force associated with the implant.

In yet another embodiment of an aspect of the invention, pre-insertion preparation of implantable material within an insertion device results in tensile delivery of the implant.

In an embodiment of another of its aspects, the invention involves an inserter device for delivering an implant into vertebral disc tissue. The implant for spinal annular repair, in some embodiments, may comprise a base member and a retention device integral with or coupled to the base member and adapted for implantation and fixation into spinal annular tissue, wherein the retention device is resistive to expulsion from the spinal annular tissue. The retention device can be integral to or separate from the base member, without limitation. The inserter device comprises an elongate tubular component having a distal end and a proximal end, the elongate tubular member having an internal diameter sized to accommodate and deliver the implant and an external diameter sized for delivery to a vertebral disc tissue treatment site. In addition, it comprises a grip component to which the tubular component is coupled, the grip component having a manual control feature, the manual control feature, when actuated, acting to advance the implant from the distal end of the elongate tubular member into a desired location with respect to the vertebral disc tissue.

In an embodiment of yet another aspect, the present invention is directed to a method of insertion of an implant into vertebral disc tissue, the implant comprising a retention device for insertion at least partially into the annulus of the disc. The retention device is resistive or has a component being resistive to expulsion from the spinal annular tissue. The method comprises the steps of: orienting the retention device in a first, delivery configuration with respect to the disc tissue; delivering the implant and the retention device into the vertebral disc tissue, the retention device being in the first, delivery configuration during at least part of the delivery; delivering the retention device at least partially into the disc nucleus; and when the retention device is at least partially in the disc nucleus, transitioning the retention device to a second, retention configuration.

In an embodiment of yet another aspect of the present invention, a method for delivering an implant into vertebral disc tissue comprises the steps of applying an insertion force to the implant at a distal portion of the implant, allowing the distal portion of the implant to which the force is applied to apply, in turn, a force, such as a tensile force, on at least one other portion of the implant, and propagating the implant by continuing to apply the insertion force, while also propagating the at least one other portion of the implant via the tensile force applied by the distal portion of the implant. In another embodiment, the implant position inside the annular cavity can be maneuvered to provide a retention configuration that has increased ability to resist expulsion from the spinal annular tissue.

In another aspect of the present invention, a method is provided for facilitating access of an implant into a vertebral disc through an aperture having a diameter and retention in the disc tissue, where the implant comprises a retention device having a first dimension that at least partially resists extrusion of the implant through the aperture when fixed in disc tissue and a second dimension of the retention device to permit insertion of the retention device through the aperture. The method comprises the steps of: identifying a diameter of the aperture; selecting a measurement of the retention device along the first dimension, the selected measurement being substantially at least three times the diameter of the aperture; and without compression of the implant or substantially modifying the diameter of the aperture, delivering the implant through the aperture.

Embodiments of devices and methods according to the present invention are closely involved with the implants they are intended to deliver. Such implants may vary in their configuration, consistent with the principles of the present invention and, without limitation, may include an implant for treatment of vertebral disc tissue, the implant introduced into vertebral disc tissue by a delivery device, the implant comprising a retention component for fixing the implant in the disc tissue, a treatment component coupled to the retention component and selected for treating the vertebral disc tissue, the retention component being reconfigurable relative to the treatment component by application of a tensile force to a distal location of the retention device, the retention component being reconfigurable from an insertion position to a retention position. An implant for use with the present invention may also include a retention device for use in implant for treatment of spinal defects, comprising, a retention element having a region comprising a center of mass or fulcrum region and at least two elongate portions projecting radially, retention element further having features for receiving applied forces, the features in positions selected relative to the center of mass or fulcrum region of the retention element such that when the applied forces are received, the retention element shifts between a first configuration for insertion and a second configuration for retention. In another embodiment, an implant for treatment of spinal defects comprises a flexible member for encouraging cellular ingrowth, having a distal portion and a proximal portion, the flexible member having an access channel extending from the proximal portion through at least a portion of the distal portion and a retention element residing at the distal portion of the flexible member and adjacent the distal end of the access channel, the retention element capable of being modulated from a first insertion position to a second retention position as acted upon by forces transmitted through the access channel. These are merely non-exhaustive examples of implants deliverable with the devices and according to methods of the present invention. Many other embodiments could also be delivered with the present invention.

An object of the present invention, of the many described herein, is to provide a kit containing one or more of the devices described herein.

### BRIEF DESCRIPTION OF THE DRAWINGS

FIG. 1 depicts a lateral view of a delivery device, in an embodiment of an aspect of the present invention, in a locked position prior to deployment.

FIG. 2 depicts the device of FIG. 1 after deployment, as used in the repair of the annulus defect post discectomy.

FIG. 3A shows a cartridge component containing implantable material or device.

FIG. 3B is a top view of the distal end of the device of FIG. 1 in a locked position with the cartridge component of FIG. 3A attached.

FIG. 4 depicts a lateral view of a trimming device, in an embodiment of an aspect of the present invention, in an open position prior to insertion of material to be cut.

FIG. 5A through FIG. 5E depict the activation of cutting blades located within the trimming device of FIG. 4.

FIGS. 6A-6D depict perspective views of an embodiment of a spinal implant delivery device according to an aspect of the present invention, including a pusher cooperating with the device for advancing the implant; the device presented in the figure, is in a locked (fault proof) position at a time point before deployment of the implant.

FIG. 7 depicts a sectional elevation of the embodiment of the spinal implant delivery device of FIG. 6 in a pre-deployment state.

FIG. 8 depicts a sectional elevation of the embodiment of the spinal implant delivery device of FIGS. 6 and 7 in a post-deployment state.

FIGS. 9A-9C depict a perspective view of a portion of another embodiment of a spinal implant of delivery device according to another aspect of the present invention, the view focusing on the distal, muzzle extremity of the delivery device and a removable cap.

FIGS. 10A-10B depict a perspective view of a portion of another embodiment of a spinal implant of delivery device according to another aspect of the present invention, the view focusing on the distal, muzzle extremity of the delivery device and a removable cap.

FIG. 11 depicts an embodiment of a spinal implant according to an aspect of the present invention.

FIGS. 12A-C depict, in perspective, elevation and plan (from below) views, an embodiment of an implant according to an aspect of the present invention, the implant of a type deliverable using one or more of the delivery devices according to the present invention.

FIG. 13 depicts, in a perspective view from a point beneath it, a retention device according to an aspect of the present invention.

FIG. 14 depicts, in an elevational view an implant being contacted by a pusher pin in accordance with an aspect of the present invention.

FIG. 15 depicts, in an elevational view from a different vantage point the implant and pusher pin shown in FIG. 14.

FIG. 16 depicts, in a sectional elevational view, an implant at a point in time prior to a muzzle-loading operation in an embodiment of the present invention.

FIG. 17 depicts, in a sectional elevational view, an implant at a point in time during a muzzle-loading operation in an embodiment of the present invention.

FIG. 18 depicts, in a sectional elevational view, an implant at a point in time following a muzzle-loading operation, in an embodiment of the present invention.

FIG. 19 shows the tip of a delivery device in an embodiment of the present invention, as shown in FIG. 18, inserted into the vertebral disc defect site, with the implant still within the delivery device, undeployed.

FIG. 20 shows the implant of FIGS. 18 and 19, fully inserted within the vertebral disc defect, with the retention device in a state of being deployed to its retention position within the nucleus of the disc, in accordance with an aspect of the present invention, and with tensile positioning elements still being engaged with the retention device.

FIG. 21 shows the implant of FIGS. 18-20, with the retention device in a state of being fully rotated, withdrawn into its fixated state within the nucleus of the disc and lodged against an inner surface of the annulus of the disc, in an embodiment of an aspect of the present invention, and with the tensile positioning elements being disengaged from the retention device.

FIG. 22 shows the implant of FIGS. 18-21, with the retention device in its fixated state or a state in which the retention device is resistive to expulsion from the spinal annular tissue with a central tension element remaining in place and a cutting element being advanced to trip the flexible portion of the implant, in accordance with an embodiment of the present invention.

FIG. 23 shows the implant of FIGS. 18-22, with the cutting element having partially completed cutting of the flexible portion of the implant, in accordance with an aspect of the present invention.

FIG. 24 shows the implant of FIGS. 18-23, with the retention device in its fixated state position and the flexible implant element, freshly cut, is pulled by the central tensioning element away from the defect site.

FIG. 25 shows the implant of FIGS. 18-24, with the retention device in its fixated state in an annulus of an otherwise repaired human disc.

### DETAILED DESCRIPTION

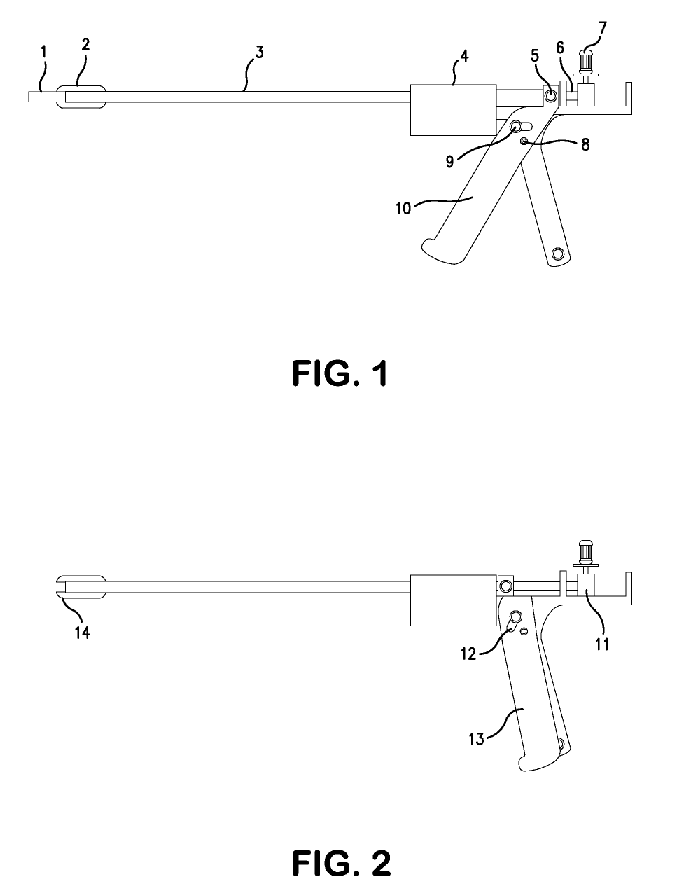

FIG. 1 depicts an embodiment of a delivery device according to the present invention, useful for inserting implants into a selected biological tissue, such as the annular and/or subannular region of an intervertebral disc. According to one such embodiment, in the form of an insertion gun providing a pistol grip, the proximal end of the device partially consists of a handle 4 and trigger 10 connected or joined by a pin joint 8 and trigger locker 9. Action on the trigger 10 results in relative motion at the distal end of the device at the tip region 1, as a displacement component 2 moves forward relative to the handle 4. Also at the proximal end of this particular embodiment is a pushing rod 6 and a front and rear stopper panel. A suture locker 7 is able to slide axially between the front and rear panel at the proximal end in part to block or enable the action of the trigger 10 through the trigger locker 9. In the orientation shown in FIG. 1, the suture locker 7 causes the trigger locker 9 to remain in a position that locks the trigger 10 in place, preventing it from being pulled back towards the handle 4.

According to an embodiment of the invention, the device has a distal terminus consisting of a slightly tapered tip region 1 with a cut slot. From the handle at the proximal end, an extendable cannula portion 3 extends along the axis of the device to connect to the distal tip 1 region. The cannula may be composed of a polymer, metallic material, or other suitable material, with a length ranging from 7 to 11 inches in certain embodiments. Also provided near the distal terminus is a displacement component 2 capable of moving axially along the distal portion of the device. Utilization of the device, from the proximal end, causes relative motion of the displacement component 2 and ejection of implant material located in the tip 1.

In the specific embodiment shown in FIGS. 1 and 2, the device accommodates an implant having a retention device in an anchor shaped like an arrowhead at the leading or distal end. The distance between the laterally extending arrowhead tips of the anchor exceeds the width of the annular defect in this example. In other embodiments, the retention device can be shaped like a wedge, umbrella or any other shape, whether symmetric or asymmetric, in which the distance between the laterally extending tips of the anchor or other portion of the retention device that resists expulsion exceeds the width of the annular defect, even up to multiples of that width, plus other shapes referred to in documents referenced in this document. As a result, the displacement component 2 is sized and shaped to dilate the annular defect in a manner that distributes pressure along the interior walls of the defect, in order to minimize or reduce abrasion or damage to the anchor during implant. The implant configuration and/or material may advantageously include aspects of the subject of International Application Serial No. PCT/US2004/043455 filed Dec. 23, 2004 designating the U.S., which is a continuation-in-part of U.S. patent application Ser. No. 10/746,563, filed Dec. 24, 2003; U.S. patent application Ser. No. 10/749,742, filed Dec. 30, 2003; U.S. patent application Ser. No. 10/848,624, filed May 17, 2004; U.S. patent application Ser. No. 11/652,763, filed Jan. 11, 2007; U.S. Provisional Patent Application No. 60/849,328, filed Oct. 3, 2006; and U.S. patent application Ser. No. Ser. No. 11/475,444, filed Jun. 26, 2006 the contents of which are incorporated herein by reference in their entirety.

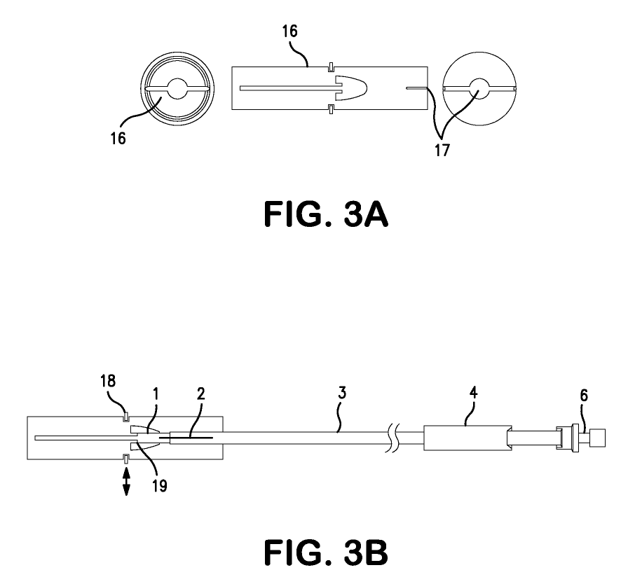

In an embodiment of a method according to the present invention, the implant material is loaded into the tip of a delivery device in order to be inserted into the desired location. FIG. 3A shows a cartridge component 16 useful for achieving this objective. According to one embodiment, the cartridge is preloaded with the implantable material from the distal end 16 and is non-permanently coupled to the distal portion of the inserter device. (FIG. 3B). At the time of use, the implant is retracted back into the tip 1 of the delivery device intra-operatively by horizontally moving a suture locker 7 at the proximal end towards the rear stopper. This action, away from the cartridge and distal region, allows the implant to be successfully transferred into the tip 1 as the sutures or threads of the implant are pulled from a rearward position. The distance between the suture locker 7 and the rear stopper is the same as that between the leading end of the implant (e.g., an anchor coupled to the implant) and the tips of the displacement component 2, ensuring correct loading into the tip 1. Following loading of the device, the cartridge may be removed.

At the point of use, the action of the suture locker 7 creates tension in the implant material as it is loaded into the device for delivery. In an embodiment as described above, the suture locker 7 pulls the implant material, by way of its elongated sutures, from the attached cartridge 16 into the tip 1 of the inserter. As a result of this pulling force, the implant is kept in a tensile state when it is loaded in the tip 1 and delivered into the disc space, avoiding buckling.

Loading the implant in the manner described herein allows one to compress the material at the time of use into a size and shape necessary for successful implantation into a vertebral disc or other suitable target. The material is not necessarily insertable in its natural or relaxed dimensions at the time of loading into the cartridge 15. By compressing or otherwise deforming the material into a narrowed shape of reduced dimension just prior to, or at a preselected time prior to, insertion, the practitioner can help ensure that the implant will be placed successfully in the chosen region. Furthermore, according to another aspect of the present invention, the implanted material need not be supplied in the ready-to-use compressed state at an earlier point in time and so stored until needed for use. Rather, the implant can be kept in a relaxed or more natural configuration, undisturbed until just prior to use. This may help better preserve the material in a condition suitable for deployment and use, and allow it to be supplied with the delivery device in a kit, as envisioned in an aspect of the present invention.

According to an embodiment of the invention, the implant emerges from the tip 1 when the suture locker 7 is brought into contact with the front stopper, to release the sutures, and the trigger 10 is subsequently pulled back 13 towards the handle, as shown in FIG. 2. At the distal terminus of the device, the displacement component moves to a forward configuration 14 upon actuation of trigger 10, pushing the implant out of the tip region 1. The displacement component 2 and the anchor at the leading end of the implant move forward unimpeded, as they are aligned with the cut slot in the tip region 1. As the implant is delivered, the displacement component 2 helps to guide the placement of an appropriate portion of the implant in the disc space and distribute the otherwise localized and deleterious forces imposed by the tips of the anchor. In addition, displacement component 2 essentially pulls the implant forward, driven by an action applied from the rear (i.e., proximally) which places the implant at least partially in a state of tension during its delivery. That is, the displacement component 2 protects the anchor and its components from compressing as the implant is loaded into and ejected from the tip 1. Upon releasing the trigger 10, the displacement component 2 is withdrawn automatically while the implant is left behind. Thus, according to an embodiment of the invention, a relatively greater force applied at the trigger and handle 13 causes a smaller force at the tip region to eject the implant. The force differential, and the manner in which the forces are applied by relative motion of portions of the practitioner's hand, allows for precision in positioning the implant, including delivering the implant out of the tip 1 and into the targeted space.

Inadvertent delivery of the implant, by pulling on the trigger 10, is avoided by the combined action of the suture locker 7 and trigger locker 9. According to an embodiment of the invention, the trigger locker 9 blocks the trigger when there is space 6 between it and the front stopper. As the suture locker 7 is moved back to the rear stopper, the implant is loaded into the tip 1 and the cartridge is subsequently removed so that the device may be used; but the trigger locker 9 continues to block the trigger 10, thereby precluding accidental deployment. Only when the suture locker is brought into contact with the front stopper panel 11 at the time of use is the trigger locker unlocked 12, allowing the trigger to be pulled 13. Thus, the associated locking mechanism of the suture locker 7 provides added safety and operational utility for the present invention. The handle and trigger grip design of the embodiment depicted in FIG. 1, or other hardware according to the present invention permitting relative motion (e.g., of parts of the practitioner's hand), allows the device to be easily manipulated or actuated, such that implantation can be properly guided.

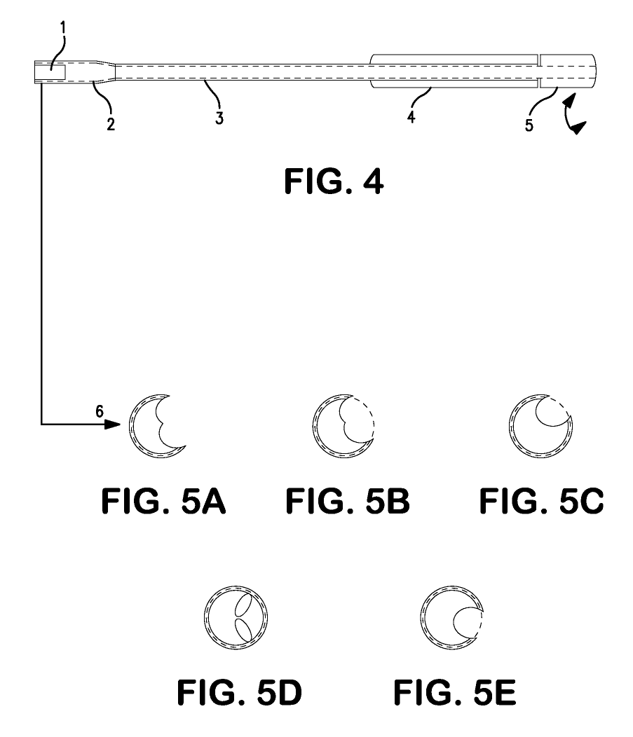

Another aspect of a kit according to the present invention involves a trimming device usable in connection with the implantable material. Embodiments of the device permit an axial approach, in a tightly confined axially-oriented space, capable of shearing the implant flush with a surface. According to an aspect of the present invention, practitioners are provided a mechanism or a means for cutting one or more targeted portions of the implant, as necessary for successful delivery. Therefore, according to one embodiment, shown in FIG. 4, a trimming device includes a proximal handle 4 and an extension part 3, terminating with a cylindrical or rounded distal region 2 constituting the site of trimming. Implantable material may be contained in the open space 1 in the internal portion of the cylindrical region, which is accessed by a partial opening of the cylindrical wall. The opening is closed by sliding a rounded wall along the perimeter of the cylinder. Such closure may be performed, for example, by rotating the handle of the device at the proximal end 5. Closure of the cylindrical region activates cutting blades 6 located in the inner cylindrical wall (FIG. 5A through FIG. 5E), which trim the enclosed implant material perpendicular to its axis. It is understood that in the present invention, the trimming device may be used in combination with the delivery device, either as an integrated or a stand alone component.

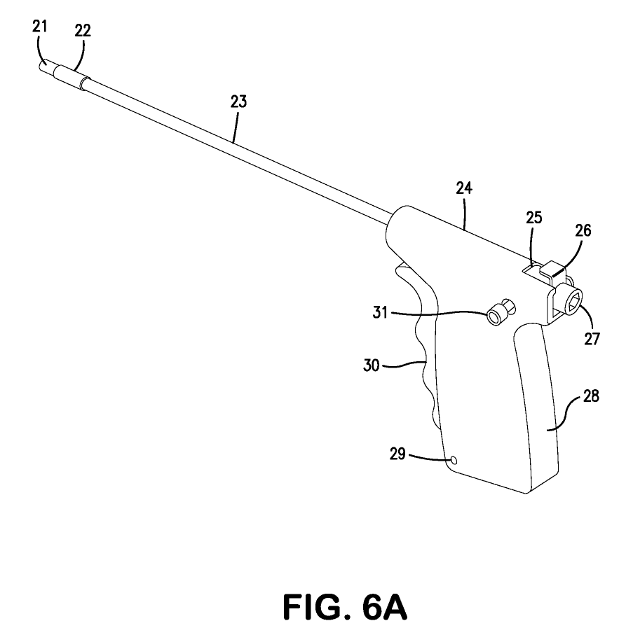

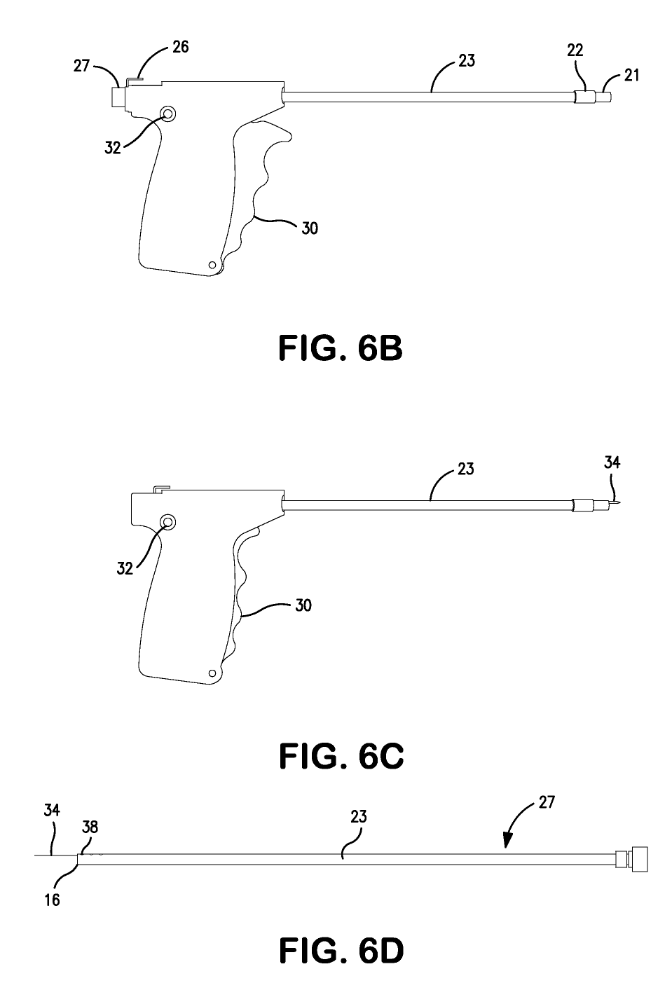

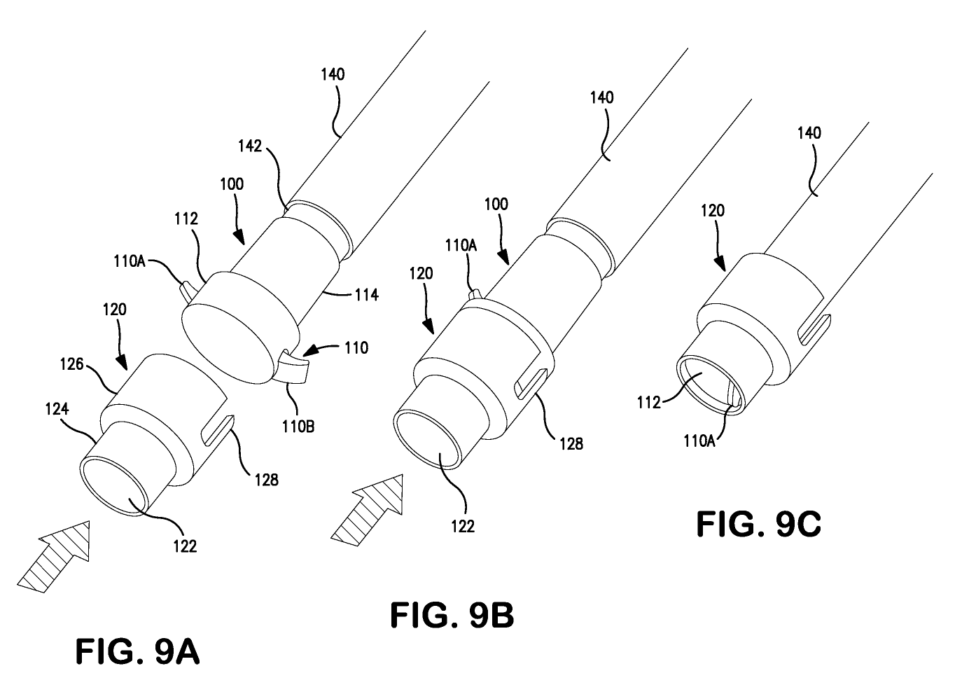

FIGS. 6A-D and FIG. 9 depict another embodiment of an insertion device according to the present invention, useful for delivering/inserting implants into a selected biological tissue region, such as the annular and/or sub annular region of the intervertebral disc. This embodiment employs many of the same principles of the gun-type insertion device described above, and includes two major components—an insertion gun 24 and a pusher 37. The pusher 37 may be involved in pre-loading the implant, in this embodiment, as well as in pushing the implant through the tube 23 of the insertion gun 24. The insertion gun 24 holds the implant, after loading from the proximal, breech end, and aids in inserting it upon pulling of the trigger 30. The breech end of the insertion device is identified by the proximal opening of the insertion device 24. In this view containing pusher 37, the breech end of which is identified by reference numeral 27. In this and the preceding embodiment, the insertion gun is breechloaded; that is, the implant is loaded into a delivery tube of the insertion gun from a proximal end, adjacent the handle. Other embodiments, such as ones that employ muzzle-loading approaches, are also encompassed by the present invention and are described below. In general, although without limitation, breech-loading implant insertion devices according to the present invention may be better suited for repeated use, with implant reloading, while muzzle-loading implant insertion devices according to the present invention may be better suited to single-use applications.

In the embodiment of FIGS. 6A-D and FIG. 9, a suture bundle (not shown) of the implant (also not shown in this view) is threaded through a distal opening of the pusher tube 23 and let to protrude through the proximal opening. The compressible implant (details of which are provided below) may be pre-loaded onto the pusher pin 12, which in one embodiment may be laser-welded to the pusher tube 15 through appropriate windows 14. The pre-loaded implant is inserted into the insertion gun 24 by undergoing compression while sliding into the compression slider 25. In one embodiment, a compression slider is a part of the gun-type insertion device, the function of which is to prepare (e.g., compress) an implant while loading and/or to slide the pusher 37 forward, as it is linked to the cam that translates the trigger pull motion to the implant advancement during delivery. The pusher 37 is pushed, which in one embodiment is with constant force, sliding the implant through the tube 23 of the insertion gun 24. The tube 23 may advantageously be made of a suitable metal, but other materials are also conceivable for the tube 23. Other examples of material of construction of tube 23 are polymers and fiber-reinforced polymers. An audible click, which arises from the springing up of spring-loaded slider 26, confirms that breechloaded pusher 27, and thereby the implant, has been loaded into the insertion gun 24 and is secure from slippage.

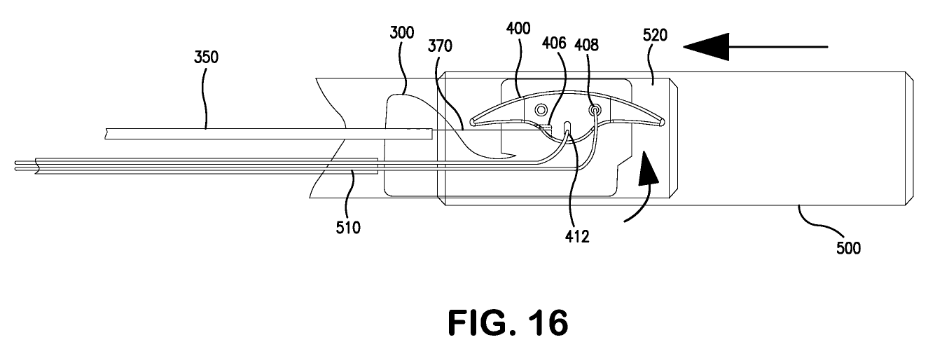

In one embodiment, the outer tube of the implant is removed after loading of the device is done, that is, after the pusher with the implant is loaded and an audible click is heard. This, in turn, exposes an inner tube and a rotation suture, which may be provided with a distinctive color or composition. The rotation suture is locked down using the suture lock knob 32, in FIGS. 6B and 6C, which may be of the form of a capstan or other suitable structure, about which the suture is wound and secured, in such a manner that there remains no slack in the rotation suture. It is also possible according to the present invention to position a portion of the suture on a suture support notch or other feature of the device at the time of securing it (e.g., spring-loaded slider 26 or in a notch at proximal end 27 of pusher 23 visible in FIGS. 6A, 7 and 8) then manipulating that suture support feature in order to introduce a preselected amount of slack into the rotation suture. That preselected amount of slack then permits an advantageous degree of discharge of the implant from the device prior to the slack being removed and the tensile force, which may include a tensile force, of the rotation suture initiating rotation of the retention device of the implant to permit insertion. In one embodiment, the slack permits the implant position inside the annular cavity to be maneuvered or otherwise re-positioned to provide a suitable retention configuration that is resistive to expulsion from the spinal annular tissue. As visible in FIGS. 16-18, rotation of the retention device may also tend to rotate the rest of the implant to a greater or lesser extent. In one embodiment, the slack in the rotation suture (or other tension element) is preselected and set to achieve the required rotation angle for the retention device of the implant, the rotation angle being between (i) an insertion or delivery configuration of the retention device when it is generally parallel with the direction of delivery and a cannula through which delivery is made, and (ii) a retention configuration of the retention device, such as when the retention device has been delivered to a disc nucleus and straddles a point of entry to resist extrusion of the implant.

The rotation of the retention device, and with it part or all of the implant, in an embodiment of an aspect of the present invention, reconfigures the implant and particularly a retention element, member, structure or other device (and which terms may be used interchangeably below), that might otherwise fit poorly, or not at all, into the defect or other insertion pathway. The retention element or device is intended to prevent the implant from extruding from the disc. Optionally the retention device can reside in the nuclear space, at the surgically created annular defect, at the surgically created annular tear or at the nucleo-annular interface or at the site of the fissures located in annulus after delivery or placement. In some embodiments, the inserted retention device substantially resides in the nuclear space. In general a retention device, device or mechanism need not comprise any fixation or anchoring structure or function but the retention device is resistive to expulsion from the spinal annular tissue. However, in some embodiments, a retention element may comprise fixation or anchoring, which generally could involve forming one or more penetrations into tissue. In particular, the retention portion is thereby rotated from a cross-section exceeding the diameter of the aperture, to a position in which its major axis is more closely aligned with the defect or other narrow insertion pathway. According to an aspect of the present invention, the major retention dimension of the retention device can be significantly in excess of the cross-sectional dimension of the defect or insertion pathway. In one embodiment the ratio of the major dimension of the retention mechanism to the width of the aperture through which it passes to the repair site is substantially 3:1. Smaller and larger ratios are also believed possible according to aspects of the present invention.

The unlock button 31 of insertion device 24 is depressed releasing the trigger 30. Once the trigger 30 is released, pulling it advances the implant through the distal tip of tube 23. Pressure is removed from the unlock button 31, while continuing to pull the trigger 30. As the trigger 30 is pulled, the implant advances and rotates at the same time. Rotation is not about the longitudinal axis of the tube 23, but in a direction of the rotation suture, which had been secured. That is, as the implant advances, the portion of the implant coupled to the rotation suture (or other tensioning element) imparts a bending-type of rotation to the implant—and thus to its retention device—by placing the major axis of the retention device in an orientation that is more or less aligned with the axis of the tube 23. The lock down of the rotation suture facilitates rotation of the implant. An audible click is heard and the unlock button 31 pops out. The side suture is released from the suture lock knob (not shown) and the inserter is withdrawn slowly, leaving the implant in position.

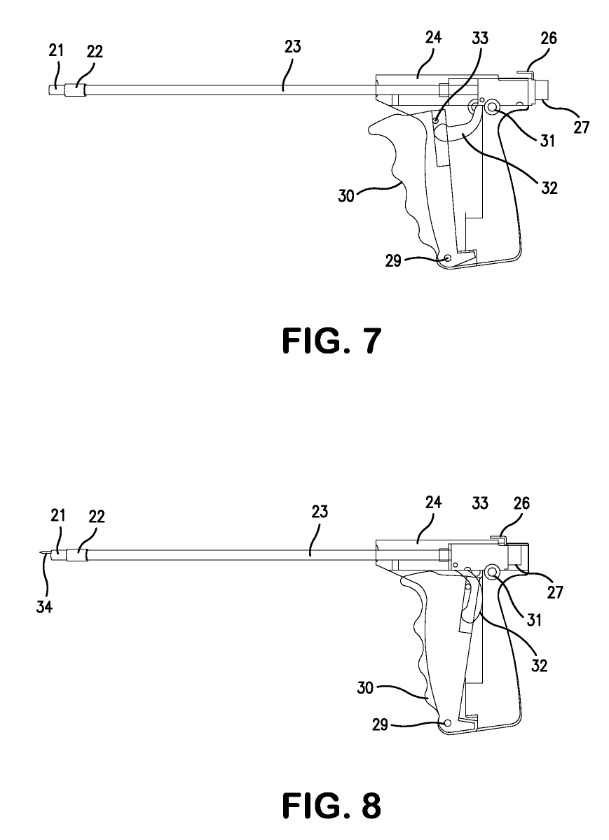

FIG. 7 depicts a sectional elevation of the embodiment of the spinal implant delivery device of FIG. 6 in a pre-deployment state. In this view, the internal mechanism and manner of mechanical operation of the insertion device 24 is more easily seen. Trigger 30 pivots, when unlocked, about hinge 29. On an internal face of trigger 30 is a point of contact with linkage 32, which is arcuate and of any suitable material, and which is rotationally borne. In one embodiment of this aspect of the present invention, the linkage 32 sits on a bar (not shown) on the compression slider. The linkage has a rectangular slit with a curved inner end that rotates on the bar on the compression slider. As the trigger is pulsed, the link is actuated which moves the compression slider, and thereby the pusher, forward. Linkage 32 is held immobile by unlock button 31. Unlock button 31 restricts the compression slider from moving which is the final motion required. As the compression slider is thus precluded from moving, pulling the trigger will apply force on the linkage which will not translated to any motion. That is, the unlock button 31 holds the compression slider immobile there, locking the trigger.

FIG. 8 depicts another sectional elevation of the embodiment of the spinal implant delivery or insertion device of FIGS. 6 and 7, in which the insertion device is depicted in a post-deployment state. In that state, trigger 30 has been fully squeezed by the user. The squeezing of the trigger 30 has pressed on the linkage, at its lower distal portion as shown in the figure, causing it to rotate, such that its upper tip moves upwardly and to the distally—and in doing so advances the pusher (not visible in this view) according to conventional principles The pusher is locked with the compression slider by means of the spring loaded slider So, as the trigger is pulled, the compression slider moves, which in turn moves along with it the pusher. The amplitude of the trigger pull is limited by the geometry of the link. The spring-loaded slider has a tiny pin at the bottom portion of its shape which the pusher engages when in the fully loaded position. This causes the spring loaded slider to spring upwards locking the pusher in place and giving an audible click, and the unlock button 31 audible snaps into the unlocked position. The unlock button 31 may in some embodiments by a generic lock which is spring-loaded and enclosed within a metal sleeve. When the trigger has reached its maximum amplitude, the compression slider gives way for the lock to spring back to its original position thereby popping the unlock button 31 with an audible click. In an embodiment of an aspect of the present invention, the audible click signals the practitioner that the insertion of the implant into the deployed position is complete. One can compare the view in FIG. 8 with the one in FIG. 7 and note that pusher 27 has moved distally to a point within the trigger of the device 24.

FIGS. 9A-9C depict, from a perspective view, loading techniques for front- or muzzle-loading of the implant into the inserter, in an embodiment of another aspect of the present invention. Once the implant is loaded using this technique or others within the scope of the present invention, the delivery mechanism may be the same.

FIGS. 9A-9C depict a perspective view of a portion of another embodiment of a spinal implant of delivery device according to another aspect of the present invention, the view focusing on the distal, muzzle extremity of the delivery device and a removable cap. This embodiment may be suitable for a single-use device. This single-use embodiment of a spinal implant and insertion device includes features that make it particularly suitable for single use, and, separately, for disposability. In a single use configuration, which in one embodiment is muzzle loaded, the insertion device with implant is deliverable with all parameters set prior to delivery so that the device may be used without substantial, or in some instances any, modification of configuration or parameters either between delivery and use, or during use.

Implant 100 has a retention device device or element 110 (which may, without limitation, comprise an anchor) embedded in a head portion 112, which may be of a matrix, as described above, or other suitable flexible material. Head portion 112 is either integral with, or coupled to, body portion 114. Upon deployment and insertion, the head portion 112, including retention device 110, reside at a treatment site, such as within the disc nucleus or sub-nucleus, the retention device being positioned across an aperture formed by a defect and being of a dimension sufficiently greater than the width of the defect that the retention device 110 prevents the implant 100 from being extruded under the pressure the vertebral disc is under during typical human activity. The body portion 114 of the implant 100 fills the defect in the region of the annulus and, if of a suitable material, permits the ingrowth of cells that repair the defect. FIG. 9 is an exploded view showing a cap 120 at a short distance from implant 100, and an insertion tube 140 having a portion of narrowed diameter 142, which in certain embodiments may create a “stop” at the point of diameter increase, to provide feedback to the practitioner.

Cap 120 includes an orifice or opening 122 at the distal end of the cap 120, and more particularly at the distal end of a portion 124 of narrowed diameter relative to a portion 126 of larger diameter. The portion 126 of larger diameter accommodates the head 112 (and retention device 110) of implant 100; the narrowed portion 124 serves to compress the implant 100 for delivery, and also permits more focused targeting of the implant for purposes of insertion. The change in diameter is useful in providing a stop or other feedback mechanism to let the practitioner know about an appropriate degree of penetration of the inserter. Cap 120, at its portion 126 of larger diameter, is provided with a groove or cutaway 128 descending distally from its proximal rim. The arrow shows the direction in which the illustrated components are moved to assemble them for later provision and use.

Referring to FIG. 9B and, again, to the arrow showing the direction in which the components are assembled, cap 120 has been placed on head 112 of implant 100. In doing so, a protruding end of retention device 110B rotates through groove 128 of cap 120; it does so because, as the cap 126 descends, the proximal rim contacts a protruding end of retention device 110A, applying a moment that causes the entire retention device 110, and the head 112 of the implant in which it is embedded, to rotate. Upon this rotation occurring, the implant is placed into a delivery or insertion configuration, in which its effective diameter is close to that of the aperture through which it must pass and significantly smaller than the width of the retention device 110 when the implant 100 and the retention device 110 are in a retention configuration. Further, in FIG. 9B, the end of retention device 110A can be seen rotating to a more proximal position, and the reduced diameter, body portion 114 of implant 100 in the vicinity of end 110 of the retention device 110 is pushed inwardly by the rotation.

FIG. 9C shows the insertion device according to this illustrated embodiment in a fully assembled state. Cap 120 is seated on distal end of tube 140. Cap 120 may be held by friction alone, as the loading steps that involve it may be performed in an operating room. In other embodiments, other arrangements may also be used consistent with this aspect of the present invention, including retention by adhesive or other mechanism. In distal orifice 122 of cap 120 can be seen end 110B of retention device 110, as well as head portion 112 of implant 100. When in position, a pusher (not visible) having a distal tip that engages retention device 110 according to other structures and methods of the present invention described here, acts upon the retention device 110, which then in turn applies a tensile force to much of the implant 100, dragging it along toward the treatment site. The implant 100 may then be made, by the pusher, to emerge through orifice 122 of cap 120. The pusher (not shown) is applied from a proximal end through the tube 140.

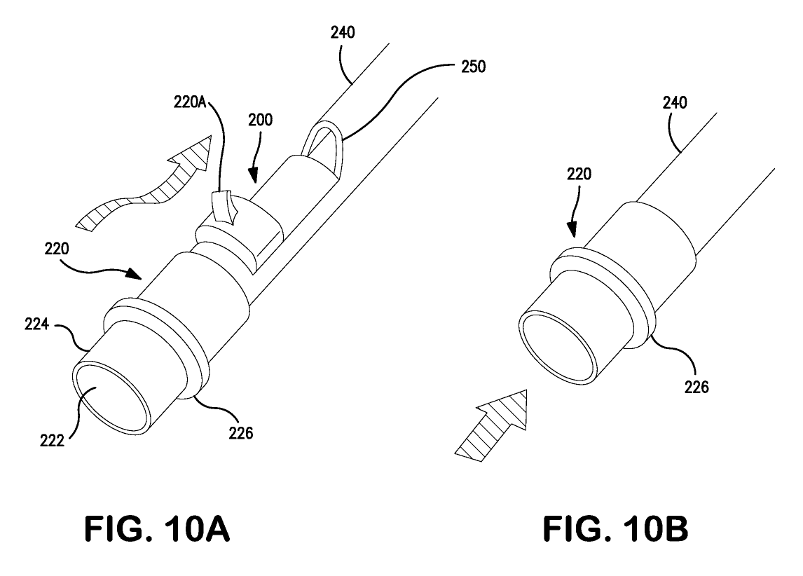

FIGS. 10A-10B depict a perspective view of a portion of another embodiment of a spinal implant of delivery device according to another aspect of the present invention, the view focusing on the distal, muzzle extremity of the delivery device and a removable cap. Like the embodiment shown in FIG. 9A-9C, this embodiment is also particularly suitable for a single-use and/or disposable implant insertion device. Cap 220, in FIG. 10A, has been placed on the distal end of tube 240 containing implant 200. As the cap 220 is progressed in the direction of the arrow, proximal rim of cap 220 acts upon end 210A of a retention device embedded in implant 200. Tube 240 is fitted with a cutaway or notch 250 that permits the retention device to rotate as cap 220 is attached. Cap 220 also includes a flange 226 protruding to a diameter a preselected magnitude larger than the primary diameter of the cap 220. The flange 226 can thus serve as a stop, providing tactile feedback to the practitioner as to the position of the insertion device relative to the vertebral disc annular defect and, then, to the nuclear space.

FIG. 10B shows the same embodiment of the insertion device of FIG. 10A, in which the cap 220 has been fully loaded onto tube 240, and in which the implant has been fully tucked away for later deployment and retention, which may be with the aid of the stop function of flange 226.

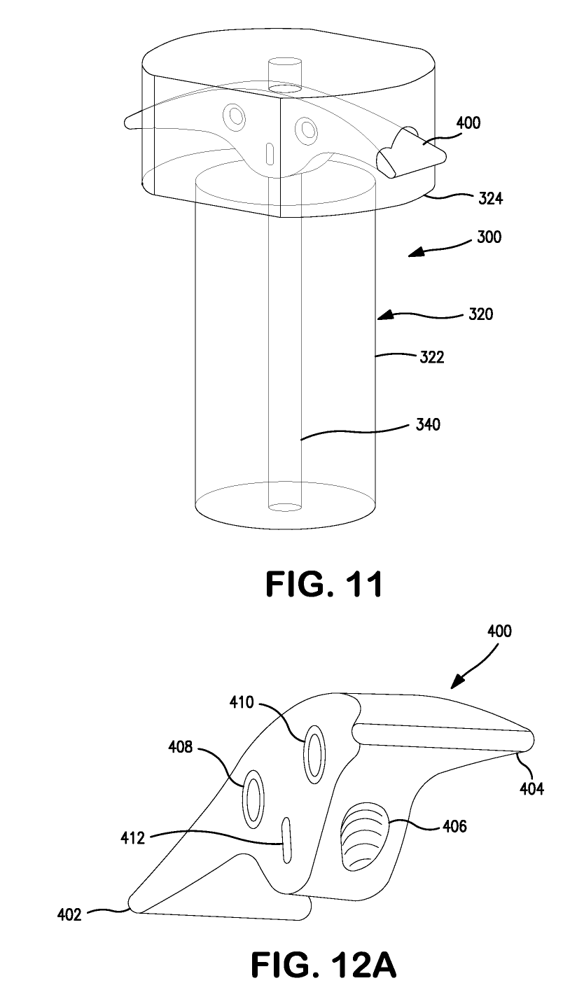

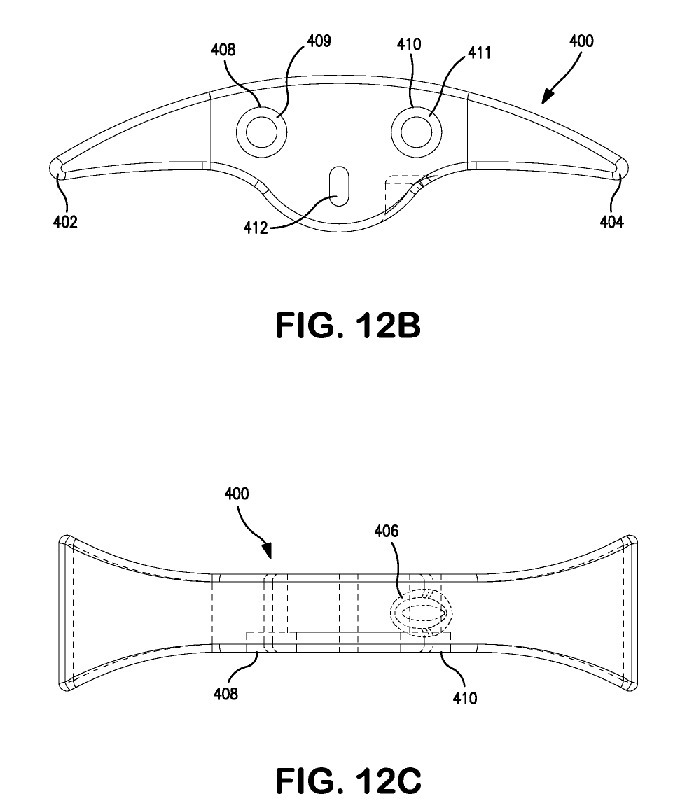

FIG. 11 depicts one embodiment of a spinal implant 300 according to an aspect of the present invention. Implant 300, in this embodiment, comprises a retention device 400 (which may be any retention structure, encompassing both non-anchoring and anchoring structures, and comprising structures of one or more components), shown in greater detail in FIGS. 12A-12C, 13 and 14. Retention device 400 is embedded in a matrix 320 which may advantageously be of a flexible material that encourages cellular ingrowth. The matrix may be any suitable material, including but not limited to those described in patent documents identified at paragraph 0043, the contents of which are hereby incorporated by reference in their entirety. In the illustrated embodiment, but without limitation, the matrix 320 may have a first, columnar portion 322, at what is a generally proximal portion of the implant in its orientation during and after insertion, and a head end portion 324 portion having a diameter greater than the columnar portion 322. The greater diameter of the head end 324 accommodates a retention element or device 400 (which may be but is not limited to an anchor) during and after insertion. The head end 324 may also be considered a part of the retention device to the extent it plays a role in resisting expulsion. In addition, the head end 324 plays a role in cushioning the impact of the retention device 400 against vertebral disc or other tissue of interest. As will be described in greater detail below, retention device 400 may rotate, under certain conditions, in a plane parallel to the axis of the columnar member. A restoring force may in some embodiments be provided by the matrix 320. Within implant 300 is a passageway 340 for the passing of structural elements such as pushers or other compressive elements and sutures or other tensile elements (not shown in this view).

FIG. 12A depicts, in a perspective view, an embodiment of a retention device 400 (which may be, but is not limited to an anchor). In the illustrated embodiment, the retention device 400 is of an anchor-type configuration. Retention device 400 may be of any suitable material. As shown, retention element 400 includes a primary axis and wing portions 402, 404 oriented parallel to that axis. Once inserted into the nucleus of a vertebral disc, as described, the retention device 400 is oriented so that its major axis straddles the aperture through which it entered, which in a typical case has been undesirably formed in the vertebral disc or other spinal tissue. In the case of treatment of a defect in the annular wall of a spinal disc, the retention device 400 will have been inserted, when in a delivery configuration more parallel to the radial direction of entry of the implant. As further described herein, when the implant reaches a desirable, final position e.g., within the nucleus of a vertebral disc (which may be flagged by feedback to the practitioner according to an aspect of the present invention) the implant is re-oriented. In its re-oriented, retention position, the extremities or wings 402, 404 of anchor 400 straddle the aperture.

Among the features of the retention element 400 may be a recess 406 or “dimple” dimensioned in width and depth for receiving the tip of a manipulation tool, such as a pusher pin (not shown). The recess 406 may be of any curved or rectilinear geometric configuration, e.g., oval/elliptical, square, round, rectangular, triangular, or combination thereof. An elliptical shape for recess 406 has been observed to orient the implant in a suitable orientation prior to delivery. The maximum transverse dimensions of recess 406 may in one embodiment, be between 0.5 to 2.5 mm and a depth of 0.5 to 1.5 mm. Recess 406 can be placed appropriately placed anywhere on the undersurface or proximal surface of the retention device. A maximum transverse dimension of recess 406, in one embodiment of this aspect of the present invention, is between 0.5 to 2.5 mm and a depth of 0.5 to 1.5 mm. Recess 406 can be placed appropriately placed anywhere on the undersurface of the retention device. In one embodiment, the recess 406 and its interaction with receiving the tip of a manipulation tool, such as a pusher pin permits the positioning of the implant inside the annular cavity to be maneuvered to provide a preferable or suitable or desired retention configuration that is resistive to expulsion from the spinal annular tissue. The degree of offset of recess 406 may, in general, be selected as a function of the dimensions of the implant, as well as other dimensions, to facilitate configuration of the implant for purposes of delivery. In addition, as shown in FIGS. 12A-12C, retention device 400 may be provided with features for coupling force-transmitting elements or members useful in reconfiguring or re-orienting the retention device 400 (and, in some embodiments, all or part of implant 300) according to aspects of the present invention. For example, holes 408 and 410, which may be through-holes in some embodiments, may receive tension elements such as sutures which can then be used to apply rotation moments, for example, about recess 406. Features 406 and 408 may in some embodiments be provided with annular rings 409 and 410 (which may be of a selected material, such as tantalum). Another feature 412, which may also but need not be a through-hole, permits tension to be applied to the center of the retention device 400 in a manner that avoids rotation of the device if the pusher pin is not positioned in recess 406. FIG. 12C shows the retention device 400 from a point below the device. In this view, it is possible to discern the generally oval shape of the recess 406 with respect to a plane parallel with the major axis of the device. The shape need not be oval, and it need not have particular dimensions; the shape and dimensions vary with the pusher pin to be used in manipulating and re-configuring the retention device 400 in use.

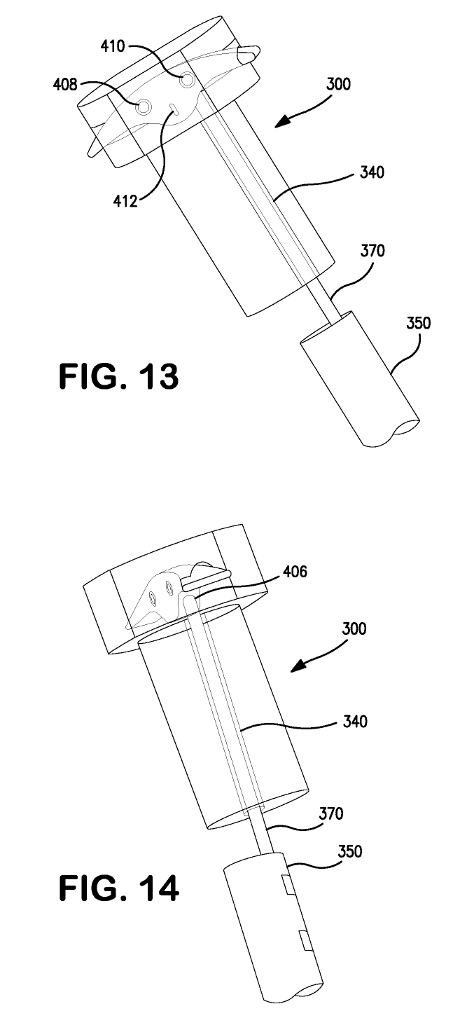

The interaction of the pusher pin with the retention element 400 is shown in greater detail in FIGS. 13 and 14. The retention element 400 may also include coupling features such as through-holes 408, 410, which permit the engagement of tensile elements such as sutures for applying orienting forces to the retention element 400. Configurations for the coupling features other than through-holes could also be used, consistent with the principles of this aspect of the present invention. In addition, the through-holes may have the salutary effect of reducing the mass of the retention element 400, while retaining strength arising from the moment of inertia of the cross-section of the retention element 400. In addition to the through-holes 408, 410, retention element 400 may include a centrally positioned through-hole 412. The through-holes 408, 410 and 412 all have positions either on-line with, or laterally offset from a center line and a center of mass of the retention device 400. Attachment of tension elements to retention device 400 at through-holes 408 or 410 can be used in coordination with a pusher pin in recess or dimple 406 to impart a moment to the retention device 400 and, with it, a desired rotation. Coupling a suture or other tension element via through-hole 412 permits a withdrawing force to be applied to the retention device 400 in a manner that would not rotate the retention device 400, unless an opposing force were also being applied at recess 406. Mechanisms other than recesses or dimples could also be used as an alternative, without departing from the scope of this aspect of the present invention.

FIGS. 13 and 14 depict, in a perspective view, a retention device being acted upon by a pusher pin 370 according to an aspect of the present invention. In FIG. 13 the retention device is viewed from the side; in FIG. 14 it is viewed at an angle. Pusher pin 370 projects from pusher tube 350, to which it may be laser-welded, for example, and engages recess 406, described above. The recess 406, in this embodiment, is offset from the center of the retention device 400, so that when a tensile force is applied to the retention device 400, e.g., by a rotation suture attached via through-hole 408 on the opposing side of the center of mass of the retention device 400, the retention device rotates.

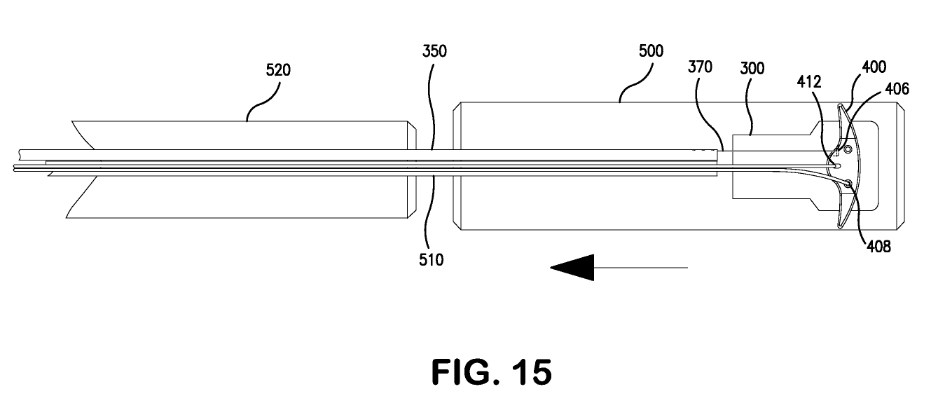

FIG. 15 depicts, in a sectional elevational view, an implant at a point in time prior to a muzzle-loading operation in an embodiment of an aspect of the present invention. This embodiment shares some similarities with the embodiment shown at FIGS. 9A-9C and 10A-10B. A cap 500 in which an implant 300 has been loaded is prepared for mounting on the distal end of delivery tube 520. The implant includes a retention device 400, having at least two manipulation sites, here an offset through-hole 408 and a central through-hole 412 at the base of the retention element 300. Prior to loading the implant 500, a first tensioning element, here in the form of a rotation suture is attached to the retention element 300 via through hole 408, and a second tensioning element for eventually securing the retention element 300 is attached to it at through-hole 412. The tensioning elements pass through a tube 510 in a proximal direction out the end of the cap 500 to delivery tube 520.

FIG. 16 depicts, in a sectional elevational view, the implant 300 and associated hardware at a later point in time during a muzzle-loading operation of the embodiment of the present invention shown in FIG. 15. In this depiction, implant 300 has been packed with a 900 bend, shown by a curving, upwardly pointing arrow, while the cap 500 is pushed in a proximal direction over the distal end of tube 520. The retention element 400 is rotated with the distal end of implant 300, leaving the tensioning element of through-hole 408 with a 900 curve in it as well.

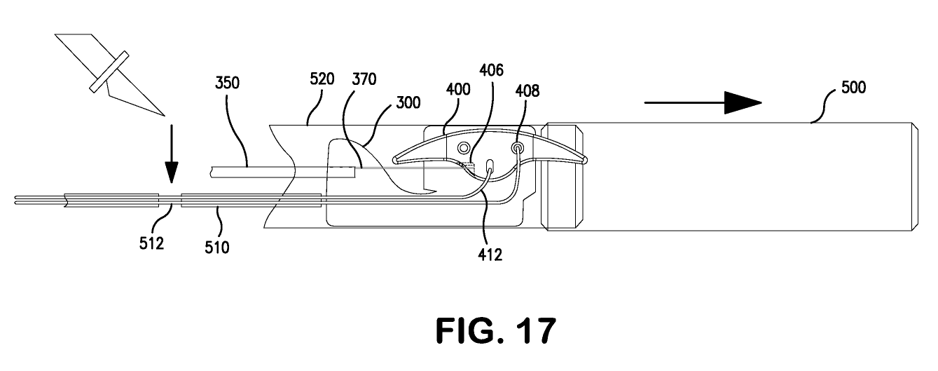

FIG. 17 depicts, in a sectional elevational view, the implant 300 and associated hardware of FIGS. 15 and 16, at a point in time following a muzzle-loading operation, in an embodiment of an aspect of the present invention. At this stage, the implant 300 has been loaded, for minimum cross-section, into the distal tip of tube 520. The cap 500 is now removed, as indicated by the rightwardly-pointing arrow. The tube 510 carrying sutures or other tensioning elements may be cut at 512 by cutting surface, as shown.

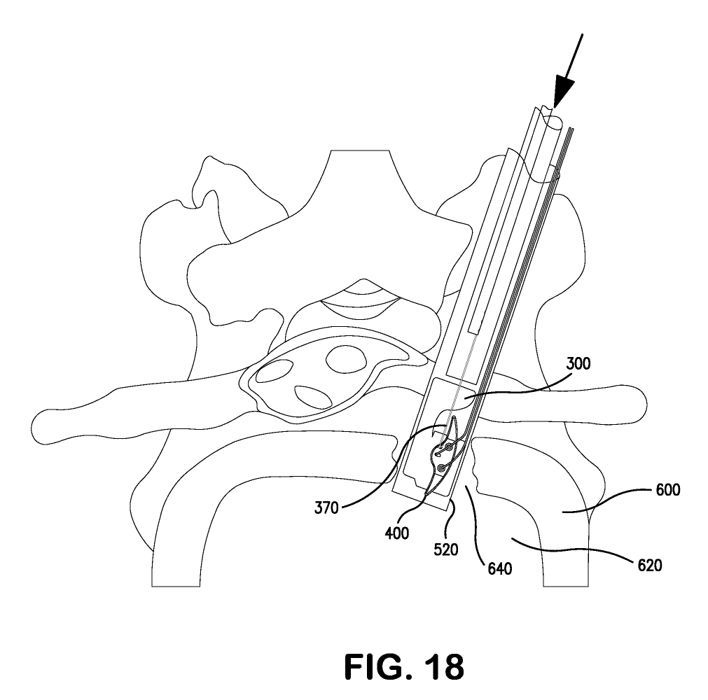

FIG. 18 depicts the distal end of a delivery device in the embodiment of the present invention shown in FIGS. 16 and 17, when inserted into the vertebral disc defect site. In the device at the stage of insertion shown in the figure, the implant 300 is still within the delivery device, undeployed. The delivery tube 520 has been inserted into a prepared insertion channel according to known methods up to vertebral disc annulus 600 and just into a defect 640 in the annulus 600, while barely penetrating disc nuclear space 620. As can be seen, the retention element 400 is oriented in accordance with an aspect of the present invention to place its major axis along the axis of travel to, and eventually through, the annular defect 640.

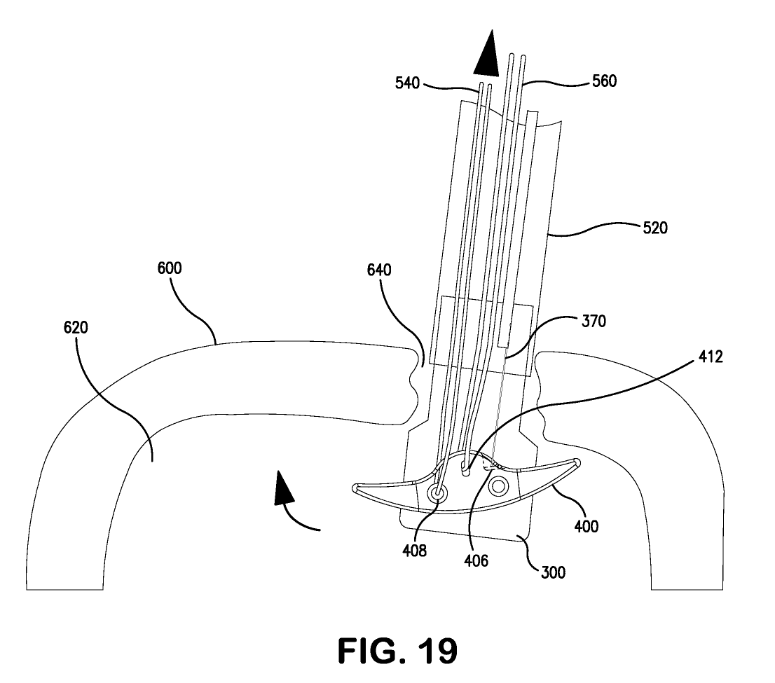

FIG. 19 shows the implant 300 of FIG. 18, fully inserted within the vertebral disc defect 640 in annulus 600. The retention element 400 in a state of being deployed within disc nucleus 620 to its retention position, in accordance with an aspect of the present invention. As shown, the retention device 400 has been partially rotated from an insertion position in which its major axis was substantially aligned with the direction of travel to and into the annular defect 640. Tensile positioning elements, in the form of rotation suture 540 and securement suture 560 are engaged with the retention device 400 in through holes 408 and 412, respectively. The arrow near the top of the figure shows that tension on rotation suture 540 applies a torque on device 400, causing it to rotate into its retention configuration, the rotation shown by the arrow immediately to the left of the retention device 400, which is curving to the left and upwardly. In one embodiment, the action of rotation suture 540 and securement (or anchor) suture 560 permits the implant position inside the annular cavity to be maneuvered to provide a suitable retention configuration that is resistive to expulsion from the spinal annular tissue. Tube 520 remains at or partially in the mouth of defect 640, the tail end of implant 130 remaining behind.

In both FIGS. 18 and 19, the implant or the part of the implant incorporating or containing the retention device can reside in the nuclear space which can be the nuclear space, or the surgically created annular defect, or the surgically created annular tear or at the nucleo-annular interface or the site of the fissures located in annulus after delivery or placement. In a preferred mode, the retention device will substantially reside in the nuclear space 620.

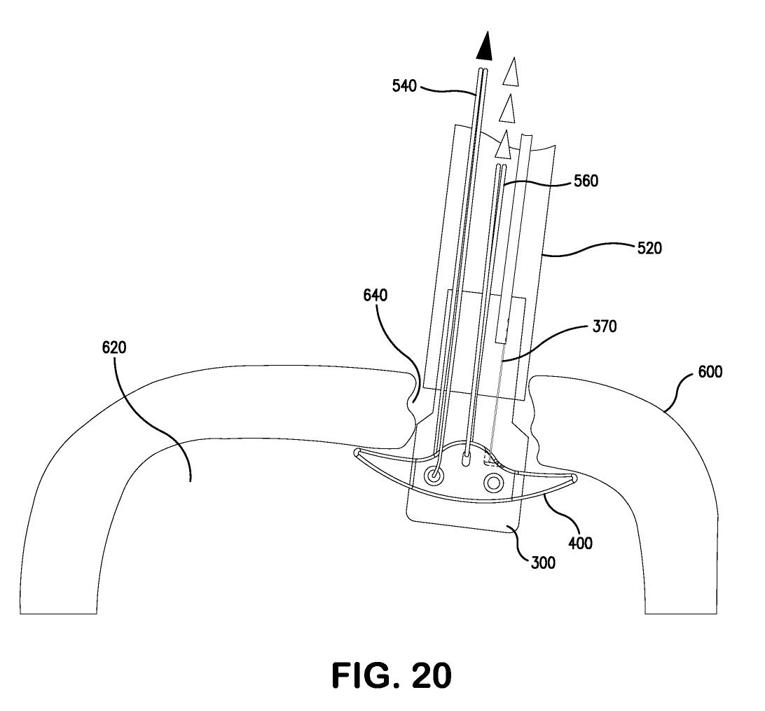

FIG. 20 shows the implant of FIGS. 18 and 19, with the retention device 400 within disc nuclear space 620 in a state of being fully rotated into its retention configuration within the nucleus of the disc. Tension on rotation suture 540 (shown by an arrow pointing upwardly) was responsible for rotating retention device 400 into a position in which both ends of the laterally protruding wing or extremity features of the retention device 400 are touching the inner surface of the annulus 600. In one embodiment, retention acts to restrain and resist the expulsion of the device from the spinal annular tissue. Arrows pointing upwardly from tensioning element 560 show the direction of a tensile force sufficient to pull the ends of retention device 400. In this view, the cap 520 is still in place and at least partially containing the proximal end of the implant 300.

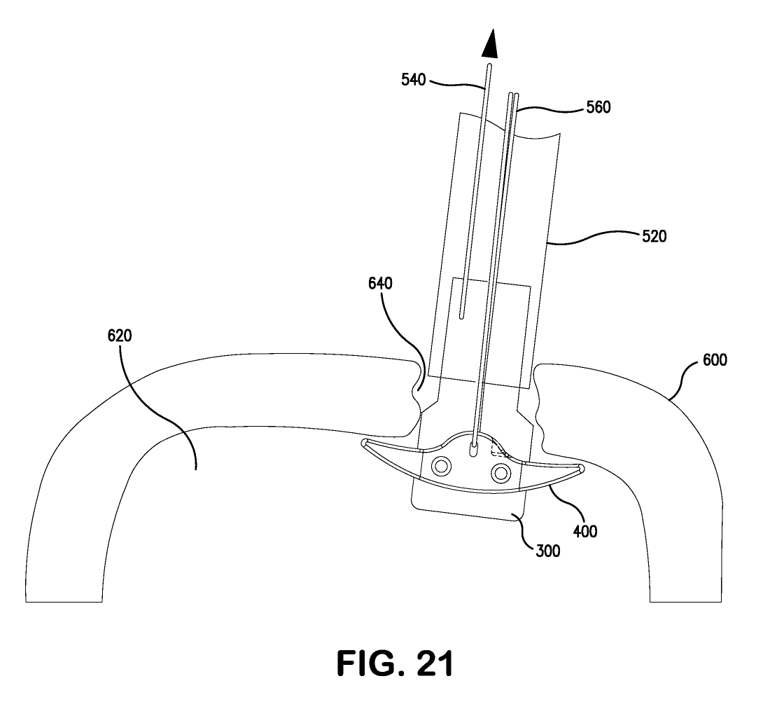

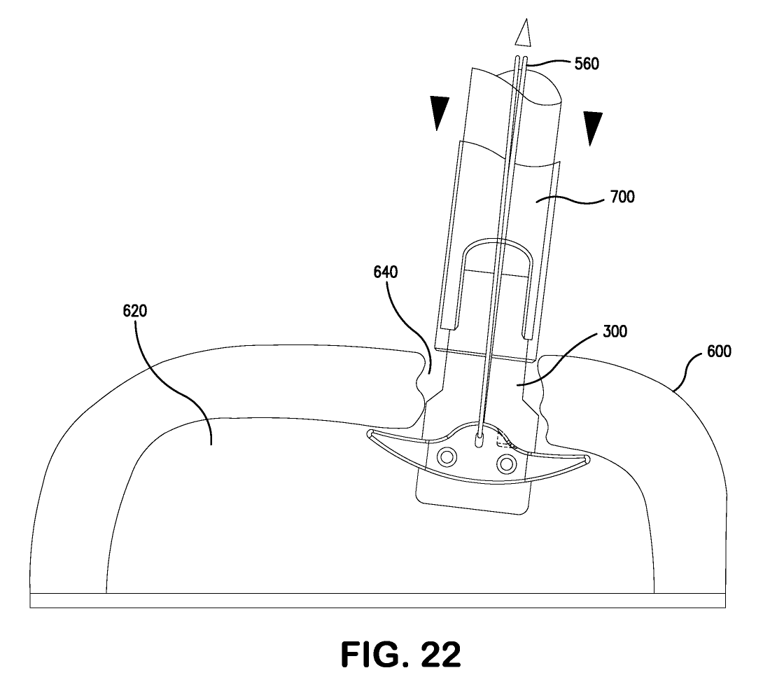

FIG. 21 is similar to FIG. 20 and shows the same embodiment, but does so at a point in time when the rotation suture 540 is being removed. In FIG. 22, the retention device 400 is in its fixated state, with a central tension element 560 remaining in place. In addition, a cutting device 700 is being advanced, on the inside of tube 520. Fixated state can be defined as a state in which the retention device is resistive or offers resistance to the expulsion from the spinal annular tissue. After being sufficiently advanced, the device 700 is positioned to trim excess material off the flexible portion of the implant 300 and seek to produce a surface that is flush with the exterior wall of annulus 600, in accordance with an embodiment of the present invention.

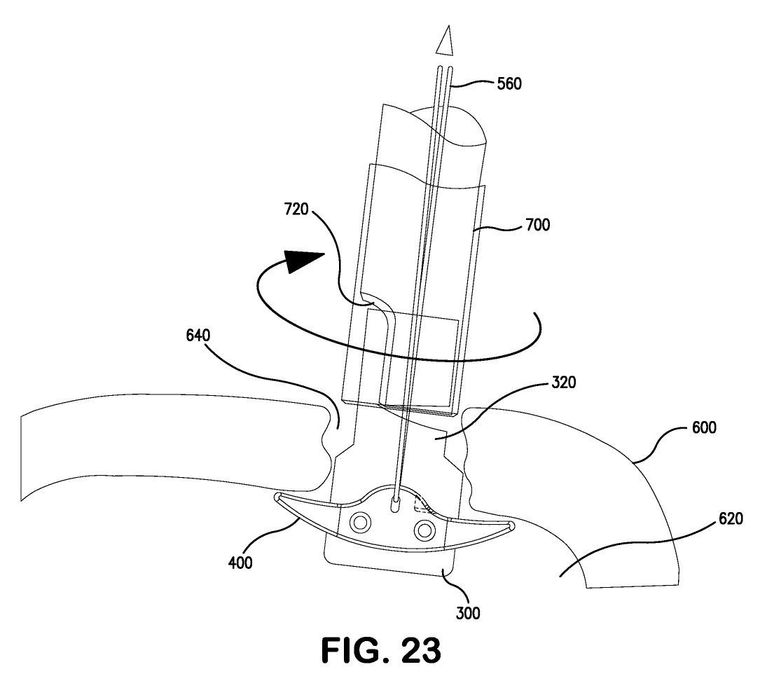

FIG. 23 shows the implant of FIGS. 19-22, with the cutting element 700 in state of nearly completely cutting the flexible portion of the implant 300, in accordance with an aspect of the present invention. In the present embodiment, cutting element 700 has a curved sharpened zone 720, and is propelled in a circular motion represented by the curved, circularly moving arrow. In doing so, the cutting element 700 slices through the implant 300, leaving a cleft 320 showing that cutting of the implant 300 is in progress. Removal of the portion of the implant 300 that is not flush reduces the risk of complications that could arise from having a free tail end of the implant protruding from the annular defect 640.

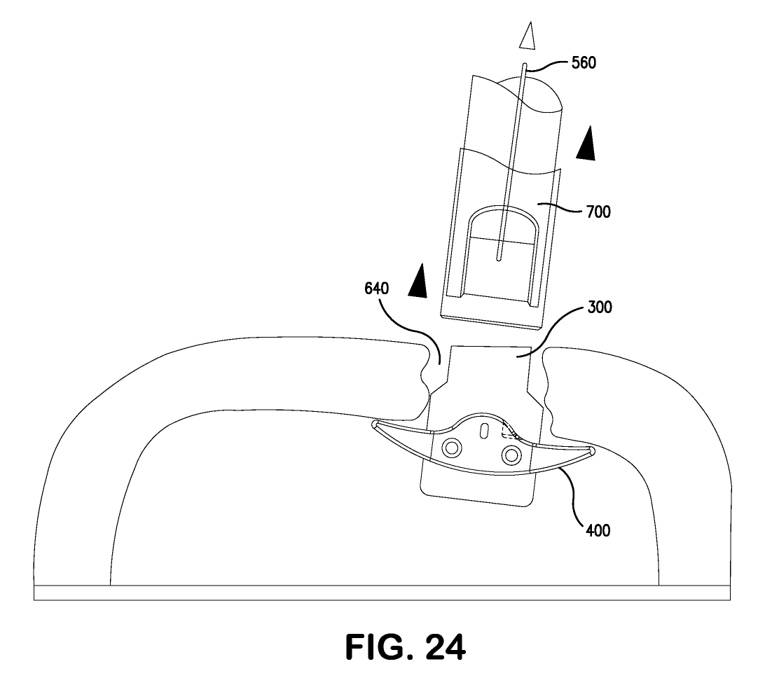

FIG. 24 shows the implant of FIGS. 19-23, with the retention device in its ultimate fixated state position. The flexible implant element, freshly cut to be substantially flush with the exterior wall, is not pulled by the central tensioning element 560 away from the defect site. Rather, the implant 300, now trimmed, and the retention device 400 are left behind to treat the annular disc repair

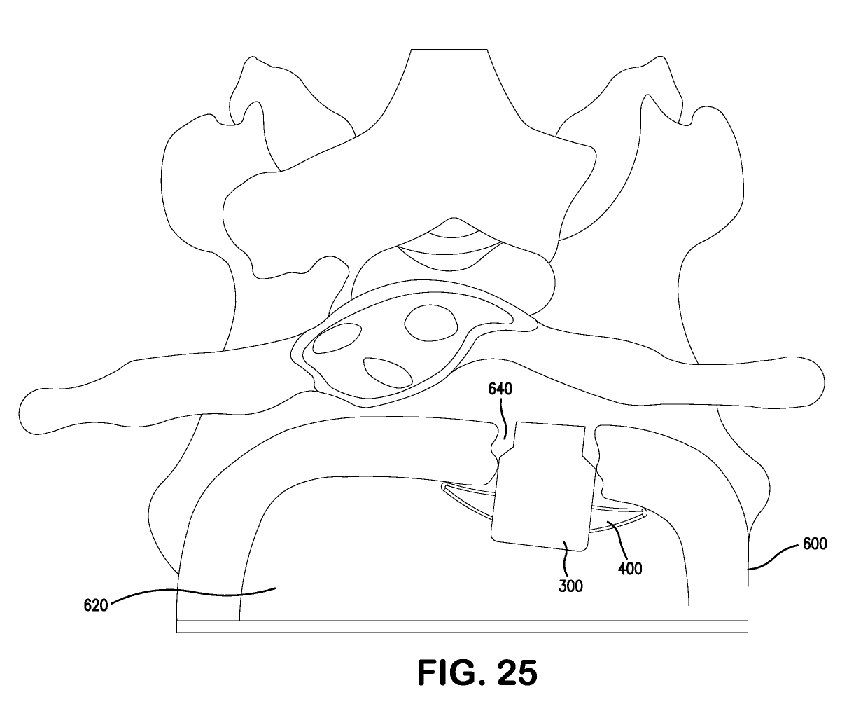

FIG. 25 shows the implant of FIGS. 19-25, with the retention device in its fixated state in nucleus 620, and in defect 640 of annulus 600, for treatment of the defect 640. The inserter of this invention delivers an implant into intervertebral disc tissue. In one embodiment, the largest transverse dimensions of the implant being is larger than the diameter of the elongate member of the inserter and the diameter of an annular defect to be treated with the implant. In one embodiment, the inserter is able to place the device in order to minimize or reduce abrasion or damage to the retention member or the anchor during implant or the vessel wall. In another embodiment, the inserter, and the interaction of the inserter with the device or the implant, permits the positioning of the implant inside the annular cavity to be maneuvered to provide a preferable or suitable or desired retention configuration that is resistive to expulsion from the spinal annular tissue.

Devices and methods for inserting implants for treatment of tissue, such as intervertebral disc tissue, reduce the profile of the implant for purposes of delivery, permitting delivery of larger implant retention structures, while tending to reduce trauma to tissue and to permit precise axial and lateral positioning of the implant and/or an associated retention structure. Other advantageous features according to the present invention may include delivery of an implant while maintaining at least a substantial portion of low-column-strength portions of the implant in tension, reconfiguring the implant to permit insertion through a diameter significantly smaller than an operative dimension of the implant when in a retention configuration following insertion, and an ability to maneuver, i.e., advance, rotate, position and reposition, the implant, by the practitioner which, among other benefits, can reduce the chances for expulsion of the implant

The approach contemplated in the present invention for insertion of the implant or repair device necessary for the repair of the annular defect post discectomy involves minimally invasive surgical techniques. Furthermore, the devices embodying aspects of the present invention described above and in the documents incorporated herein by reference may find application at all vertebral segments of the spine or other suitable tissue. The various aspects of the present invention do not necessarily all need to be used together, but may in appropriate instances be used or exchanged with other aspects of the present invention. For example, although vertebral implant insertion guns according to an aspect of the present invention may advantageously be used to deliver implants according to other aspects of the present invention, the vertebral implant insertion guns according to the present invention might also be usable with other implants, including those that are as-yet undeveloped. Similarly, implants according to aspects of the present invention may well be deliverable using insertion devices other than those according to aspects of the present invention, including ones that are as yet undeveloped. Other objects, advantages and embodiments of the various aspects of the present invention will be apparent to those skilled in the field of the invention and are within the scope of the description and the accompanying figures. For example, but without limitation, structural or functional elements might be arranged, or method steps reordered, consistent with the present invention. Similarly, principles according to the present invention, and systems and methods that embody them, could be applied to other examples, which, even if not specifically described here in detail, would nevertheless be within the scope of the present invention.

What is claimed is:

1. An inserter device for delivering an implant into vertebral disc tissue, comprising:
an elongate tubular component having a distal end and a proximal end, the elongate tubular member having an internal diameter sized to accommodate and deliver the implant and an external diameter sized for delivery to a vertebral disc tissue treatment site;
a grip component to which the tubular component is coupled, the grip component having a manual control feature, the manual control feature, when actuated, acting to advance the implant from the distal end of the elongate tubular member into a desired location with respect to the vertebral disc tissue.

2. The inserter device according to claim 1, wherein the grip component comprises a pistol-type grip.

3. The inserter device according to claim 1, in which the manual control feature comprises a trigger.

4. The inserter device according to claim 1, further comprising a grip for holding a proximal portion of a flexible element, the distal portion of the flexible element coupled to a distal portion of the implant.

5. The inserter device according to claim 1, wherein the elongate tubular member is also dimensioned to accommodate a pusher for advancing the implant along the length of the tubular member.

6. The device according to claim 1, in which the tubular member comprises a stopper for impeding propagation of the inserter.

7. The device according to claim 1, in which the device is manipulable by a user to perform at least one of the group of actions consisting of advancing, rotating, positioning, and repositioning the implant.

8. A method of insertion of an implant into vertebral disc tissue, the implant comprising a retention device for insertion at least partially into the annulus of the disc, the method comprising the steps of:
orienting the retention device in a first, delivery configuration with respect to the disc tissue;
delivering the implant and the retention device into the vertebral disc tissue, the retention device being in the first, delivery configuration during at least part of the delivery;
delivering the retention device at least partially into the disc nucleus;
when the retention device is at least partially in the disc nucleus, transitioning the retention device to a second, retention configuration.

9. The method according to claim 8, wherein the delivery is performed with an inserter operating with a cannula and wherein the first delivery configuration comprises a position in which the retention device is rotated substantially toward an orientation parallel with the cannula. to the.

10. The method according to claim 9 wherein configuring and/or reconfiguring of the implant is performed using a least one of a compressive element and a tensile element.

11. The method according to claim 10, wherein configuring and/or reconfiguring of the implant is performed using a compressive element at a first selected time and a tensile element at a second selected time.

12. The method according to claim 11, wherein the first selected time and second selected time may at least partially temporally overlap.

13. The method according to claim 9, wherein the rotated state of the retention device is effected using a tensile element.

14. The method according to claim 10, wherein the tensile element is coupled at a distal end to the retention device and at a proximal end to a delivery device.

15. The method according to claim 11, wherein the tensile element comprises a filament.

16. The method according to claim 12, wherein the filament comprises suture material.

17. The method according to claim 8, wherein the retention device comprises an anchor.

18. A method for delivering an implant into vertebral disc tissue comprising the steps of:
applying an insertion force to the implant at a distal portion of the implant;
allowing the distal portion of the implant to which the force is applied to apply, in turn, a tensile force on at least one other portion of the implant;
propagating the implant by continuing to apply the insertion force, while also propagating the at least one other portion of the implant via the tensile force applied by the distal portion of the implant.

19. The method according to claim 18, wherein the at least one other portion of the implant comprises material having insufficient column strength to permit insertion of the portion of the at least one other portion of the implant without buckling.

20. The method according to claim 18, in which the at least one other portion of the implant that is propagated by the tensile force is kept substantially free of bunching.

21. A method for facilitating access of an implant into a vertebral disc through an aperture having a diameter and retention in the disc tissue, the implant comprising a retention device having a first dimension that at least partially resists extrusion of the implant through the aperture when fixed in disc tissue and a second dimension of the retention device to permit insertion of the retention device through the aperture, the method comprising the steps of:
identifying a diameter of the aperture;
selecting a measurement of the retention device along the first dimension, the selected measurement being substantially at least three times the diameter of the aperture; and
without compression of the implant or substantially modifying the diameter of the aperture, delivering the implant through the aperture

22. The method according to claim 21, further comprising the step of
selecting a measurement of the retention device along the second dimension to be less than or equal to the diameter of the aperture.

23. The method according to claim 22, wherein the ratio of the measurement along the first dimension to the measurement along the second division is greater than or equal to three.

24. The method according to claim 22, wherein the ratio of the measurement along the first dimension to the measurement along the second division is greater than or equal to three.

25. The method according to claim 22, wherein the ratio of the measurement along the first dimension to the measurement along the second division is greater than or equal to four.

26. The method according to claim 22, wherein the ratio of the measurement along the first dimension to the measurement along the second division is greater than or equal to two.
